(19) United States
(12) Patent Application Publication
(10) Pub. No.: US 2009/0157183 A1
(43) Pub. Date: Jun. 18, 2009

(54) Method of treating spinal disorders

(75) Inventor: John K. Song, Chicago, IL (US)

Correspondence Address: JOHN K. SONG, 474 NORTH LAKE SHORE DR., APT. # 4508, CHICAGO, IL, 60611-6465, US

(73) Assignee: John K. Song, Chicago, IL (US)

(21) Appl. No.: 11/998,534

(22) Filed: Dec. 1, 2007

(51) Int. Cl. A61F 2/44; A61N 1/00; A61B 5/04

(52) U.S. Cl. 623/17.11; 607/117; 600/377

(57) ABSTRACT

A method for treating disorders of the spine by placing a stimulating electrode into the disc space. The method of treatment and means of applying the technique are given.

### FIELD OF THE INVENTION

The present invention relates to a method for treating disorders of the spine using electrical stimulation.

### BACKGROUND OF THE INVENTION

Complaints related to the spine make up a significant portion of annual visits to health care providers and lost productivity. Current treatments include both surgical and non-surgical means. Surgical treatments include common procedures such as laminectomy, discectomy, spinal fusions, and more recently disc and nucleus replacement. Non-surgical treatments include physical therapy, medications, and injections.

Unfortunately, non-surgical treatments are time consuming, do not often work, and must be repeated indefinitely. Surgical treatments can be more effective but only a small portion of patients with spine problems qualify for spinal surgery.

One of the most common problems of the spine is low back pain. It is unclear often times where the pain is coming from and what is causing it and unfortunately many patients simply have to suffer with this problem.

One treatment for spinal problems that has been around for some time is called spinal cord stimulation. This involves the placement of a configuration of simple electrical contacts spaced along an electrode or wire which is connected to battery powered electronics (or a pulse generator). The electronics send out continuous or intermittent signals to one or more of these electrical contacts to modulate pain related to the spine. Typically, this procedure works best for pain in the extremity but can also work for pain along the spinal axis.

The procedure involves either open or percutaneous placement of the electrodes in the dorsal epidural space usually in the cervical or thoracic spine. Although the exact mechanism of action is unknown, it may work by blocking pain signals from getting to the brain.

The method of the present invention proposes to treat complaints related to the spine by applying stimulating electrodes to the disc itself (rather than placing them in the epidural space) as that can be a source of pain.

One technology that is superficially similar but distinctly different is radiofrequency ablation (e.g. Sharkey et al, U.S. Pat. No. 7,282,061). In this technique, an electrode is placed into the disc space and a radiofrequency generator is used to provide energy to the tip of the electrode which is placed into the disc space. The electrode is not implanted and the purpose of the applied energy is to burn or heat the disc to shrink it or destroy any pain fibers. The method of the present invention does not aim to heat or destroy the disc material and rather than permanently destroying any pain fibers, the method of the present invention aims to merely modulate or mask the pain from the disc or otherwise influence the disc material non-destructively.

U.S. Patent Documents

| | | |
|---|---|---|
| 2006/0241717 | Oct-06 | Whitehurst et al. |
| 7110820 | Sep-06 | Tcheng et al. |
| 2006/0217782 | Sep-06 | Boveja Ct al. |
| 2006/0173522 | Aug-06 | Osorio |
| 2006/0122660 | Jun-06 | Boveja et al. |
| 2006/0129205 | Jun-06 | Boveja et al. |
| 2006/0106430 | May- | Fowler et al. |
| | 06 | |
| 2006/0106431 | May- | Wyler et al. |
| | 06 | |
| 7039470 | May- | Wessman |
| | 06 | |
| 7039470 | May- | Wessman |
| | 06 | |
| 7024247 | Apr-06 | Gliner et al. |
| 7010351 | Mar-06 | Firlik et al. |
| 6999819 | Feb-06 | Swoyer et al. |
| 2006/0025829 | Feb-06 | Armstrong et al. |
| 7006859 | Feb-06 | Osorio et al. |
| 6990377 | Jan-06 | Gliner et al. |
| 2006/0015153 | Jan-06 | Gliner et al. |
| 6959215 | Oct-05 | Gliner et al. |
| 6941171 | Sep-05 | Mann et al. |
| 6944497 | Sep-05 | Stypulkowski |
| 6944501 | Sep-05 | Pless |
| 6937894 | Aug-05 | Isaac et al. |
| 6934580 | Aug-05 | Osorio et al. |
| 2005/0182453 | Aug-05 | Whitehurst |
| 6920359 | Jul-05 | Meadows et al. |
| 2005/0149146 | Jul-05 | Boveja et al. |
| 2005/0154425 | Jul-05 | Boveja et al. |
| 2005/0154426 | Jul-05 | Boveja et al. |
| 6907293 | Jun-05 | Grill et al. |
| 2005/0143787 | Jun-05 | Boveja et al. |
| 6907296 | Jun-05 | Doan et al. |
| 2005/0119712 | Jun-05 | Shafer |
| 6892097 | May- | Holsheimer |
| | 05 | |
| 6895280 | May- | Meadows et al. |
| | 05 | |
| 2005/0096701 | May- | Donovan et al. |
| | 05 | |
| 2005/0113882 | May- | Cameron et al. |
| | 05 | |
| 2005/0080463 | Apr-05 | Stahmann et al. |
| 2005/0090756 | Apr-05 | Wolf et al. |
| 2005/0075679 | Apr-05 | Gliner et al. |
| 2005/0075680 | Apr-05 | Lowry et al. |
| 6868288 | Mar-05 | Thompson |
| 2005/0055063 | Mar-05 | Loeb et al. |
| 6873872 | Mar-05 | Gluckman et al. |
| 2005/0070971 | Mar-05 | Fowler et al. |
| 2005/0033378 | Feb-05 | Sheffield et al. |
| 6839594 | Jan-05 | Cohen et al. |
| 2005/0004620 | Jan-05 | Singhal et al. |
| 2005/0015129 | Jan-05 | Mische |
| 2005/0021104 | Jan-05 | DiLorenzo |
| 2005/0021105 | Jan-05 | Firlik et al. |
| 2005/0021106 | Jan-05 | Firlik et al. |
| 2005/0021107 | Jan-05 | Firlik et al. |
| 2005/0021118 | Jan-05 | Genau et al. |
| 2004/0243205 | Dec-04 | Keravel et al. |
| 2004/0249422 | Dec-04 | Gliner et al. |
| 2004/0236388 | Nov-04 | Gielen et al. |
| 6810286 | Oct-04 | Donovan et al. |
| 2004/0215287 | Oct-04 | Swoyer et al. |
| 6788975 | Sep-04 | Whitehurst et al. |
| 6795737 | Sep-04 | Gielen et al. |
| 2004/0176831 | Sep-04 | Gliner et al. |
| 2004/0181263 | Sep-04 | Balzer et al. |
| 6782292 | Aug-04 | Whitehurst |
| 2004/0158298 | Aug-04 | Gliner |
| 6764498 | Jul-04 | Mische |
| 2004/0131998 | Jul-04 | Marom et al. |
| 2004/0138550 | Jul-04 | Hartlep et al. |
| 2004/0111127 | Jun-04 | Gliner et al. |
| 6735474 | May-04 | Loeb et al. |
| 6735475 | May-04 | Whitehurst et al. |
| 2004/0088024 | May-04 | Firlik et al. |
| 2004/0088024 | May- | Firlik et al. |
| | 04 | |
| 2004/0092809 | May- | Decharms |
| | 04 | |
| 2004/0102828 | May- | Lowry et al. |
| | 04 | |
| 6725094 | Apr-04 | Saberski |
| 2004/0073270 | Apr-04 | Firlik et al. |
| 2004/0082847 | Apr-04 | McDermott |
| 6708064 | Mar-04 | Rezai |
| 6704956 | Mar-04 | Riley et al. |
| 6704956 | Mar-04 | Riley et al. |
| 6687525 | Feb-04 | Llinas |
| 6690974 | Feb-04 | Archer et al. |
| 6684105 | Jan-04 | Cohen et al. |
| 6658299 | Dec-03 | Dobelle |
| 6665562 | Dec-03 | Gluckman et al. |
| 6650943 | Nov-03 | Whitehurst et al. |
| 6652449 | Nov-03 | Gross et al. |
| 6647296 | Nov-03 | Fischell et al. |
| 6645203 | Nov-03 | Sharkey et al. |
| 6633780 | Oct-03 | Berger |
| 2003/0187490 | Oct-03 | Gliner |
| 2003/0187491 | Oct-03 | Greenberg et al. |

### SUMMARY OF THE INVENTION

While the invention has been shown and described with reference to certain preferred embodiments, it will be understood by those skilled in the art that various changes and modifications in form and detail may be made therein without departing from the spirit and scope of the invention, as defined by the appended claims.

It is an objective of the present invention to provide a novel method of treating spinal complaints using commercially available electrodes.

It is an objective of the present invention to provide a method of placing one or more electrodes into the disc space itself via a trocar.

It is an objective of the present invention to provide a means of treating common spinal complaints including but not limited to: neck pain, back pain, lumbago, cervicalgia, spondylosis, annular tear, degenerative disc disease, degenerative joint disease, herniated disc, disc bulge, “black disc”, etc.

It is an objective of the present invention to describe a device comprising an electrode with electrical contacts which is placed into the disc space incorporating a feature to help retain the electrode within the disc space.

Variations in electrode design and configuration are possible. Multiple electrical contacts can be spaced along a single electrode and multiple electrodes can be used. The preferred embodiment involves placing the electrodes from a posterolateral approach into the disc space from either a unilateral or bilateral approach utilizing a cannulated trocar. Other methods of placement can include simple open surgical placement, placement from a direct lateral or direct anterior approach.

As an example only, with no intention of limitation of scope, the following is given as a possible sequence of events:
1) A disc space of interest is identified for the surgical procedure.
2) A cannulated trocar with central stylet is placed through the skin into the disc space from a posterior/posterolateral approach near the spinal neural foramen.
3) The central stylet is removed
4) An appropriately selected electrode is placed through the trocar into the disc space. This electrode can be situated either to cross the midline or two or more electrodes can be used. The trocar is removed leaving the electrode in place.
5) The electrode(s) are connected to an implantable pulse generator.
6) The pulse generator is implanted under the skin
7) All incisions are closed.

Use of the device and the means of treating spinal disorders would then involve programming of various parameters including frequency, amplitude, timing, and determining which electrodes to use.

Alternatively, another embodiment of the present invention might involve placement of a self contained device which fits entirely within the disc space and delivers the electrical current via charge stored in a battery or some form of capacitor. Recharging of the device could come from either an external inductance coil or via a mechanism within the disc space which generates electricity for storage via movement of the spine and/or changes in pressure in the disc space.

### BRIEF DESCRIPTION OF THE DRAWINGS

FIG. 1. Shows an axial via of a spinal vertebra sectioned through the disc space with an electrode in place.

### DETAILED DESCRIPTION OF THE DRAWINGS

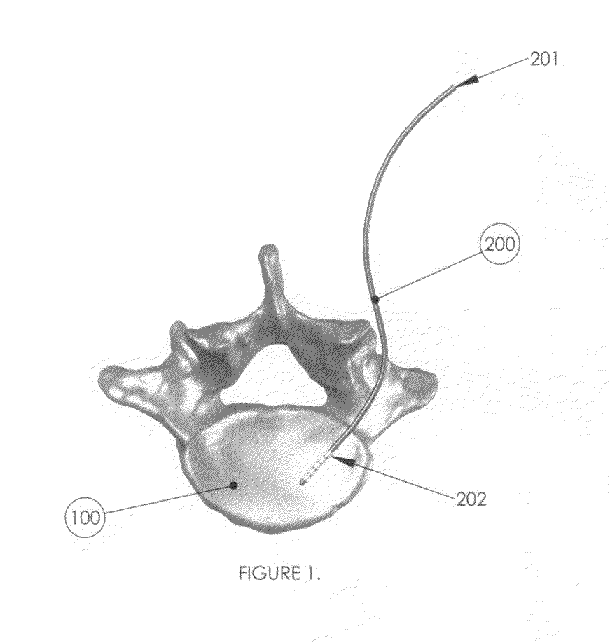

FIG. 1 illustrates one configuration of how a simple electrode might be placed within the disc space. Item 100 is the vertebral body. Item 200 is a sample electrode placed within the disc space adjacent to 100. A unilateral electrode is depicted for simplicity; however, it is understood that this electrode may also cross the midline. In addition, multiple electrodes introduced from any angle may be used. As used in the context of this discussion, “electrode” refers to the entire device which is connected to the power source and “electrical contact” refers to a feature of the electrode. Item 201 shows the proximal end of the electrode which is intended to be connected to suitable electronics and a power supply. Item 202 shows a sample electrical contact although the shape, number, and distribution of the electrical contact(s) may vary.

What is claimed is:

1. A method of treating spinal disorders by placing one or more electrodes into a spinal disc and applying an electrical current between said electrodes by means of a pulse generator.

2. The method of claim 1 where the electrodes and/or the pulse generator are permanent implants.

3. The method of claim 1 where the electrodes and/or the pulse generator are temporary implants.

4. The method of claim 1 where the pulse generator delivers a continuous electrical current.

5. The method of claim 1 where the pulse generator delivers an intermittent electrical current.

6. The method of claim 1 where the pulse generator delivers a programmable and customizable current between one or more electrodes.

7. The method of claim 1 where multiple electrodes are used.

8. A device of the present invention comprising an electrode with one or more electrical contacts which is placed into the disc space incorporating a feature for retaining the electrode in the disc space.

9. The device of claim 8 where the retaining feature is a barb.

10. The device of claim 8 where the retaining feature is a hook.

11. The device of claim 8 where the retaining feature is a fin.

12. The device of claim 8 where the retaining feature is a flange.

13. The device of claim 8 where the retaining feature is an expanded leading tip which provides pullout resistance.

14. A method of placing an electrode into the disc space by through the use of a cannulated tube as follows: A cannulated tube with a central stylet is placed through the skin into the disc space, the central stylet is removed, one or more electrodes are placed though the cannulated tube into the disc space, the cannulated tube is removed.

15. The method of claim 14 where the electrode(s) is/are placed unilaterally.

16. The method of claim 14 where the electrode(s) is/are placed bilaterally.

17. The method of claim 14 where the electrode(s) is/are placed crossing the midline.

18. A device for treating spinal disorders which can be implanted and delivers a non-destructive electrical current to the disc space.

19. Device of claim 18 where the device is connected to a power source external to the disc space.

20. Device of claim 18 where the device and power source are contained within the disc space

21. Device of claim 18 where normal spinal motion and forces provides a source of electrical current.
